(19) United States
(12) Patent Application Publication
(10) Pub. No.: US 2025/0000885 A1
(43) Pub. Date: Jan. 2, 2025

(54) COMPOSITION COMPRISING TWO OR MORE OLIGOSACCHARIDES

(71) Applicant: Children's Hospital Los Angeles, Los Angeles, CA (US)

(72) Inventors: Michael I. GORAN, Los Angeles, CA (US); Bradley Scott PETERSON, Los Angeles, CA (US); Lars BODE, San Diego, CA (US); Ravi BANSAL, Los Angeles, CA (US)

(73) Assignee: Children's Hospital Los Angeles, Los Angeles, CA (US)

(21) Appl. No.: 18/830,190

(22) Filed: Sep. 10, 2024

Related U.S. Application Data
(63) Continuation of application No. PCT/US2023/032509, filed on Sep. 12, 2023.
(60) Provisional application No. 63/581,896, filed on Sep. 11, 2023.
(60) Provisional application No. 63/581,896, filed on Sep. 11, 2023.
(60) Provisional application No. 63/405,815, filed on Sep. 12, 2022.

(51) Int. Cl. A61K 31/702; A61P 25/00

(52) U.S. Cl. CPC A61K 31/702; A61P 25/00

(57) ABSTRACT

A composition, comprising: two or more oligosaccharides selected from the group consisting of 2′-fucosyllactose (2′FL), 3-fucosyllactose (3FL), and 3′-sialyllactose (3′SL) is disclosed.

### RELATED APPLICATIONS

The present application claims priority to U.S. Provisional Application No. 63/581,896, filed on Sep. 11, 2023, the disclosure of which is hereby incorporated by reference in its entirety. The present application is a continuation of PCT Application No. PCT/US2023/032509, filed on Sep. 12, 2023, which claims priority to U.S. Provisional Application No. 63/581,896, filed on Sep. 11, 2023 and U.S. Provisional Application No. 63/405,815, filed on Sep. 12, 2022, the disclosures of all of which applications are hereby incorporated by reference in their entireties.

### FIELD OF THE INVENTION

The present disclosure generally relates to a composition comprising two or more oligosaccharides selected from 2′FL, 3FL, and 3′SL.

### BACKGROUND OF THE INVENTION

Human milk is the standard for infant nutrition, as research suggests that a greater frequency and duration of breastfeeding benefits infant health. However, evidence in support of the benefits of breastfeeding for some infant outcomes has been inconsistent, perhaps in part because of differences in milk composition over the course of lactation, between women, and across populations. While human milk composition is complex and presents methodological challenges for research, it is critically important to consider the variation in concentrations of milk components to fully understand the benefits of breastfeeding as part of a biological system, the mother-milk-infant ‘triad’.

Human milk oligosaccharides (HMOs) are structurally diverse, complex carbohydrates that may confer some of the proposed benefits of human milk on infant outcomes. HMO biosynthesis follows a basic blueprint that begins with a lactose molecule that is elongated and either fucosylated or sialylated to create various subgroups. These slight structural differences impart diverse physiological functions for HMOs in infant health. Our research team reported that higher concentrations of HMO, 2′-fucosyllactose (2′FL), in maternal milk at 1-month predicted better cognitive development at 24-months of age. Animal studies have shown that feeding 2′FL during lactation enhanced learning and memory in rats, whereas feeding sialylated HMOs, 3′-sialyllactose (3′SL) and 6′-sialyllactose (6′SL), improved cognitive performance in piglets. HMOs are thought to promote early brain development through at least two mechanisms: 1) HMOs are prebiotics that shape the development of the gut microbiome and its production of metabolites that affect brain functions; and 2) HMOs are a source of sialic acid (SA), an essential nutrient in ganglioside formation and myelination, key components of cortical gray matter and developing white matter. The specific effects of individual HMOs on infant brain development, however, are unknown.

What is needed is a composition, such as an infant formula, that includes digestible complex carbohydrates that can improve brain development.

### BRIEF DESCRIPTION OF THE DRAWINGS

Features of the present disclosure are illustrated by way of example and not limited in the following figure(s), in which like numerals indicate like elements, in which:

FIG. 1 is a participant flow chart;

FIG. 2 shows statistical maps of 2′FL exposure at 1-month of age with newborn MRI measures;

FIG. 3 shows statistical maps of 3FL exposure at 1-month of age with newborn MRI measures; and

FIG. 4 shows statistical maps of 3′SL exposure at 1-month of age with newborn MRI measures.

### SUMMARY OF THE INVENTION

In an aspect, there is disclosed a composition, comprising: two or more oligosaccharides selected from the group consisting of 2′-fucosyllactose (2′FL), 3-fucosyllactose (3FL), and 3′-sialyllactose (3′SL).

In an aspect, there is disclosed a method of increasing brain development in a patient in need thereof, comprising: providing a patient with a composition comprising two or more oligosaccharides selected from the group consisting of 2′-fucosyllactose (2′FL), 3-fucosyllactose (3FL), and 3′-sialyllactose (3′SL).

Additional features and advantages of various embodiments will be set forth, in part, in the description that follows, and will, in part, be apparent from the description, or can be learned by the practice of various embodiments. The objectives and other advantages of various embodiments will be realized and attained by means of the elements and combinations particularly pointed out in the description herein.

### DETAILED DESCRIPTION OF THE INVENTION

For simplicity and illustrative purposes, the present disclosure is described by referring mainly to an example thereof. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure.

Additionally, the elements depicted in the accompanying figures may include additional components and some of the components described in those figures may be removed and/or modified without departing from scopes of the present disclosure. Further, the elements depicted in the figures may not be drawn to scale and thus, the elements may have sizes and/or configurations that differ from those shown in the figures.

It is to be understood that both the foregoing general description and the following detailed description are exemplary and explanatory only, and are intended to provide an explanation of various embodiments of the present teachings.

The term “human milk oligosaccharides (HMO)” refers generally to a number of complex carbohydrates found in human milk. Among the monomers of milk oligosaccharides are D-glucose (Glc), D-galactose (Gal), N-acetylglucosamine (GlcNAC), L-fucose (Fuc), and sialic acid [N-acetylneuraminic acid (NeuAc)]. Elongation may be achieved by attachment of GlcNAc residues linked in β1-3 or β1-4 linkage to a Gal residue followed by further addition of Gal in a β-1-3 or β-1-4 bond. Most HMOs carry lactose at their reducing end. From these monomers, a large number of core structures may be formed.

The term “isolated,” when applied to an oligosaccharide, denotes that the oligosaccharide is essentially free of other milk components with which it is associated in the natural state, i.e., in human breast milk. It can be in, for example, a dry or aqueous solution.

The term “purified” denotes that an oligosaccharide has been separated at least in part from other components of human breast milk. Particular oligosaccharides can be purified individually or a combination of oligosaccharides can be purified away from at least one other component of milk. In some embodiments, the oligosaccharide can be at least 85% pure, optionally at least 95% pure, and optionally at least 99% pure.

An “individual,” “subject,” or “patient” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, a mammal is a human.

In its broad and varied embodiments, disclosed herein is a composition, comprising two or more oligosaccharides, such as, 2′-fucosyllactose (2′FL), 3-fucosyllactose (3FL), and 3′-sialyllactose (3′SL), or their variants, isomers, analogs, and derivatives thereof. The data reveal that fucosylated and sialylated HMOs differentially associate with indices of tissue microstructure and rCBF, suggesting specific roles for 2′FL, 3FL, and 3′SL in early brain maturation.

Further, derivatives of oligosaccharides of disclosure may be made by covalent linking an oligosaccharide to any other chemical compound or polymer, using methods known in the art of organic and synthetic chemistry or through enzymatic methods. These derivatives include, but are not limited to, attaching or covalently linking the oligosaccharides of the disclosure to other oligosaccharides, amino acids, polypeptides, and nucleic acids.

Further, isomer, analog and derivatives of the oligosaccharides of the disclosure may be made by substituting a sugar residue within an oligosaccharide disclosed herein with a sugar analog. For example, galactose may be substituted with its analogs, including but not limited to 2-desoxy-D-galactose, 2-desoxy-2-fluoro-D-galactose and 2-desoxy-2-amino-D-galactose. For example, glucose may be substituted with its analogs, including, but not limited to, 2-Deoxy-D-glucose, 2,2-difluoro-deoxy-D-glucose, 2-deoxy-2-fluoro-2-iodo-D-glucose, 1-O-methyl-D-glucose, 2-O-methyl-D-glucose, 2-deoxy-2-chloro-D-glucose, 2-deoxy-2-bromo-D-glucose, 3-O-11C-methyl-D-glucose, 6-deoxy-D-glucose, 6-deoxy-6-fluoro-D-glucose, and 6-deoxy-6-iodo-D-glucose, and 2-deoxy-2-18F-fluoro-D-glucose. For example, N-acetylglucosamine may be substituted with its analogs, N-acetylglucosaminylasparagine, N-acetylglucosamine 6-sulfate, N-acetylglucosamine-1-phosphate, N-acetylglucosamine 6-phosphate, methyl-2-acetamido-2-deoxy-D-glucopyranoside, N-acetylglucosaminitol, N-bromoacetylglucosamine, 2-acetamido-1,3,6-tri-O-acetyl-4-deoxy-4-fluoroglucopyranose, N-acetylglucosamine thiazoline, N-fluoroacetyl-D-glucosamine, 2-acetamido-2-deoxy-D-glucono-(1,5)-lactone, and 3-acetamido-3,6-dideoxyglucose.

Furthermore, isomers of an oligosaccharide of the disclosure may be obtained based on a chiral center, such that D-glucose as a six-member ring can exist either as α-D-glucopyranose or β-D-glucopyranose, depending on the orientation of the hydroxyl group at the C-1 position with respect to the rest of the ring. Similarly, D-galactose, N-acetylglucosamine and sialic acid rings may exist in either α- or β-conformation. The isomers of an oligosaccharide may differ based on α- or β-position of the acetal functional groups. For example, the glycosidic linkage between galactose and glucose may be α-acetal functional group instead of β-acetal functional group, e.g., an α-1,4 glycosidic linkage instead of a β1,4 glycosidic linkage. Thus, a number of isomers of the oligosaccharide may exist based on the orientation of the hydroxyl-group at the C-1 or C-2 position of the six-member rings.

Since modification by sialic acid introduces a negative charge in the form of a carboxyl-group (Coo-), other monosaccharides also contain carboxyl-groups and may substitute for sialic acid in an oligosaccharide of the disclosure variant, isomer, analog and derivatives thereof. These sugars could be glucuronic acid, galacturonic acid, iduronic acids, 3-Deoxy-D-manno-oct-2-ulosonic acid, neuraminic acid, or any other carboxyl-group containing monosaccharides or derivatives thereof.

Variants, analogs and derivatives including its isomers and metabolites can be produced by modifying an oligosaccharide disclosed herein through substitutions, modifications, and conjugations that preserve the biological activity of improving or increasing brain development in a subject, including infant human subjects.

The oligosaccharides of the disclosure can be derived using any of a number of sources and methods known to those of skill in the art. For example, oligosaccharides disclosed herein can be purified from human milk using methods known in the art. One such method for extraction of oligosaccharides from pooled mother's milk entails the centrifugation of milk at 5,000×g for 30 minutes at 4° C. and fat removal. Ethanol is then added to precipitate proteins. After centrifugation to sediment precipitated protein, the resulting solvent is collected and dried by rotary evaporation. The resulting material is adjusted to the appropriate pH of 6.8 with phosphate buffer and β-galactosidase is added. After incubation, the solution is extracted with chloroform-methanol, and the aqueous layer is collected. Monosaccharides and disaccharides are removed by selective adsorption of the oligosaccharides using solid phase extraction with graphitized nonporous carbon cartridges. The retained oligosaccharides can be eluted with water-acetonitrile (60:40) with 0.01% trifluoroacetic acid. Individual oligosaccharides can be further separated using methods known in the art such as capillary electrophoresis, HPLC (e.g., high-performance anion-exchange chromatography with pulsed amperometric detection; HPAEC-PAD), and thin layer chromatography. This process can provide substantially pure oligosaccharides being at least 50% pure of other material present in the raw milk, or at least 60%, 70%, 80%, 85%, 90%, 95%, 98%, 99% or 100% pure. In yet another embodiment, the process can provide purification of individual oligosaccharide “types” (e.g., 3′SL or 2FL). The method can provide a specific oligosaccharide being at least 50% pure of other material present in the raw milk, or at least 60%, 70%, 80%, 85%, 90%, 95%, 98%, 99% or 100% pure of any other oligosaccharide or other component of raw milk.

Alternatively, enzymatic methods can be used to synthesize the oligosaccharides of the disclosure. In general, any oligosaccharide biosynthetic enzyme or catabolic enzyme (with the reaction running in reverse) that converts a substrate into any of oligosaccharide structures disclosed herein (or their intermediates) may be used.

6′SL, which is almost structurally identical to 3′SL, had no significant effects. This suggests that not all HMOs contribute significantly to a given neurodevelopmental outcome.

2′FL exposure associated inversely with FA values throughout most of the cortical mantle. It also associated positively with MD values in posterior portions of developing white matter. Prior human studies have shown that FA values in the cortical mantle decline with age during late gestation. Animal studies have shown that this decline in FA values is accompanied by increasing MD values and derives from an age-related increase in dendritic arborization within the cortical mantle. FA declines and MD increases with increasing dendritic arborization in cortical gray matter because dendrites and axons arborize without directional preference. 2′FL exposure at 1-month can increase dendritic arborization throughout the cortical mantle. Dendritic arborization and synaptogenesis are the microstructural basis for learning and memory. Increased dendritic arborization and synapse formation could therefore account for improved cognitive outcomes associated with 2′FL exposure found in this same cohort at 24-months of age. 2′FL exposure also associated inversely with rCBF in gray matter of frontal, temporal, and parietal cortices, in similar locations where we observed inverse associations of 2′FL with FA values. Because rCBF is tightly coupled to cellular metabolism, it is considered a valid surrogate measure of local metabolism. Reduced rCBF in cortical gray matter therefore represent lower metabolism. The presence of lower metabolism in the same cortical locations of greater dendritic arborization and synaptic density suggests that the neural circuits forming in early infancy may be metabolically more efficient in direct proportion to the quantity of 2′FL in milk consumed during early infancy. The formation of more efficient neural circuits would account for the improved cognitive outcomes associated with 2′FL ingestion in this same cohort of infants. The possibility that 2′FL promotes the formation of more efficient neural circuits and thereby improves cognitive development is consistent with the widely held theory that metabolically more efficient neural circuits are the cellular basis for higher intelligence.

3FL and 3′SL exposures associated positively with FA values in developing white matter. They also associated inversely with MD values in these same general locations. Studies in infants have shown that FA values in developing axonal tracts increase rapidly with age from birth to 12-months due to an age-related increase in myelination that improves white matter integrity. As FA values increase in developing axonal tracts, MD values decrease in these same locations because myelination, as well as changes in axon density and orientation, constrain the movement of water parallel to the long axis of nerve fiber bundles. Myelination supports faster information processing and improved information transfer throughout the brain, critical for higher-order cognitive functions.

3FL and 3′SL also associated positively with rCBF values in developing white matter in similar locations where we observed their positive associations with FA values, consistent with findings in adults. The FA and MD associations that suggest 3FL and 3′SL may promote more rapid myelination in developing white matter therefore also suggest that the concomitant associated increase in rCBF in these white matter regions may be driven by a significant metabolic demand on oligodendrocytes, which produce myelin. These interpretations of findings underscore the value of acquiring multiple imaging modalities in the same brains. Each modality provides unique information about tissue characteristics at each brain location, and their use in combination better constrains the interpretation of possible cellular determinants for the observed effects.

The identification of 3FL and 3′SL as having distinct roles in brain development at 1-month of age is particularly relevant, because 3FL and 3′SL were the only HMOs to increase across lactation. In our comprehensive analysis of HMO changes over lactation, most HMO concentrations declined sharply within the first 6-months. 2′FL did not significantly change over 24-months while only two others significantly increased over time, 3FL (10-fold increase) and 3′SL (2-fold increase).

Specific HMOs may influence tissue microstructure in the infant brain through several mechanisms. HMOs are prebiotics for the gut microbiome which produces metabolites that may affect early brain development. Treatment with 2′FL and 3FL in vitro increases the abundance of Lactobacillus and Bacteroides, gut microbes that have been associated with better cognitive development in infants. Greater abundance of Lactobacillus and Bacteroides produces greater concentrations of short chain fatty acids (SCFAs, metabolites that cross the blood-brain barrier for use as energy substrates to support cellular metabolism during early brain development). SCFAs also affect cell signaling and the induction of tyrosine hydroxylase that increases neurotransmitter synthesis and release. 2′FL and 3FL can enhance growth of distinct gut bacteria, which produce SCFAs and other metabolites that may support more rapid dendritic arborization, synaptogenesis, and myelination.

Another potential mechanism whereby HMOs may produce the observed associations with MRI measures is by lending structural support for maturational events in the newborn brain. Sialylated HMOs, 3′SL and 6′SL, contain SA, an essential component of brain gangliosides and polysialylated neural cell adhesion molecule (NCAM). Gangliosides and polysialylated NCAM play critical roles in cell-to-cell interactions, neuronal outgrowth, neuronal migration, and modulation of synaptic activity, ultimately supporting improved memory formation. For example, piglets fed 3′SL, 6′SL, and SA alone performed better on learning and memory tasks compared to controls, which was attributed to increased ganglioside concentrations in prefrontal cortex and corpus callosum. Associations of 3′SL and total HMO-bound Sia exposures with newborn MRI measures were observed in similar locations of the prefrontal cortex and corpus callosum. Gangliosides and polysialylated NCAM are also receptors for myelin-associated glycoprotein expressed on the innermost myelin membrane adjacent to the axon's surface, where it enhances axon-myelin stability. Exposure to 3′SL and total HMO-bound Sia may therefore enhance gangliosides and polysialylated NCAM for axon-myelin interactions and axon stability, accounting for the observed 3′SL and total HMO-bound Sia associations with MRI indices of white matter maturation in our sample. However, the structural isomer 6′SL, which also carries SA in a different linkage, was not associated with MRI measures, indicating that the effects are not simply based on the provision of SA, but are in fact structurally-dependent.

The composition can include two or more oligosaccharides in suitable amounts to improve brain development. Examples of suitable amounts of the total concentration of the two or more oligosaccharides of the invention include, but are not limited to: an amount of about at least 30 μM, at least 300 μM, at least 600 μM, at least 800 μM, greater than 800 μM, in the range of approximately 10 μM-10,000 μM, approximately 600-1500 μM or approximately 500-800 μM dosage forms; or compositions containing active ingredient (the total concentration of the two or more oligosaccharides of the invention) of about at least 38.7 mg/L, at least 387 mg/L, in the range of approximately 12.9 mg to 12.9 g per liter, approximately 774 mg/L to 1,935 mg/L, approximately 645 mg/L to 1,032 mg/L, or approximately 200-500 mg/L of the two or more oligosaccharides or derivative thereof with the balance made up from non-toxic carrier may be prepared. In some embodiments, these amounts or ranges may vary by about 10%. In other embodiments, the amounts or ranges may vary by about 20%. In still other embodiments, these amounts or ranges may vary by about 25%. Methods for preparation of these compositions are known to those skilled in the art.

The disclosure contemplates non-naturally occurring formulations of 2FL, 3FL, and/or 3′SL. For example, the formulation can lack one or more components found in human milk such as, but not limited to proteins (e.g., antibodies, lactoferrin, mucins, growth factors), lipids (e.g., DHA, AA, FAA) and/or lactose. In some embodiments, the compositions of the disclosure comprises, consists essentially of or consists of 2FL, 3FL, and/or 3′SL, wherein the 2FL, 3FL, and/or 3′SL is at least 50%, 60%, 70%, 80%, 90%, 95%, 98%, 99% or 100% free of any other human milk component. In some embodiments, the composition comprising, consisting essentially of or consisting of 2FL, 3FL, and/or 3′SL is in the form of a nutraceutical or pharmaceutical. In some embodiment, a composition comprising, consisting essentially of or consisting of 2FL, 3FL, and/or 3′SL is in a freeze dried preparation such that it can be added to a liquid or beverage.

The composition can include other nutrients chosen from fat, protein, other carbohydrates, mineral, vitamin, and combinations thereof. The other carbohydrate is any carbohydrate that is different from 2FL, 3FL, and 3′SL.

The composition can be in the form of a powder. The powder can be reconstituted prior to consumption. The powder can be formulated into a capsule or a tablet. In an aspect, the composition can be ready-to-feed liquid, or a concentrate that is diluted prior to consumption.

The concentration of the two or more oligosaccharides of the invention in the formulation will depend on absorption, inactivation and excretion rates of the two or more oligosaccharides of the invention, the dosage schedule, and amount administered as well as other factors known to those of skill in the art. The concentrations of the two or more oligosaccharides of the invention are effective for delivery of an amount, upon administration, that treats, prevents, or ameliorates one or more of the symptoms of diseases or disorders to be treated.

In the formulations, two or more oligosaccharides of the invention can also be mixed with other mammalian or plant proteins. For example, mammalian proteins include proteins from mammalian milk (e.g., either intact or partial protein hydrolysates of whole or fractionated mammalian milk). Plant proteins include intact protein or protein hydrolysate from pea, soy, almond, and/or rice proteins. In the formulation, the weight fraction of the two or more oligosaccharides of the invention may be dissolved, suspended, dispersed or otherwise mixed in a selected carrier at an effective concentration such that the treated condition is relieved, prevented, or one or more symptoms are ameliorated.

In one embodiment, the formulation of the invention is an enteral formulation. Enteral formulations of the invention may be embodied in an infant formula, breast milk, water, juices, health drinks or health foods, or baby food for infants, children or adults. Additionally, enteral formulations of the invention may be embodied in a nutritional supplement suitable for any age.

In the formulations of the invention, two or more oligosaccharides can also be mixed with other mammalian or plant proteins. For example, mammalian proteins include proteins from mammalian milk e.g., either intact or partial protein hydrolysates of whole or fractionated mammalian milk. Plant proteins include intact protein or protein hydrolysate from pea, soy, almond, and/or rice proteins.

In a further embodiment, the formulation of the invention may be added to any liquid for consumption. Liquids include, but are not limited to, water or juices.

In another embodiment, the formulation of the invention is used to supplement or fortify the mother's own milk or human donor milk (human milk fortifier) with two or more oligosaccharides and/or their derivatives, isomers, and analogs.

The composition can include pharmaceutically acceptable excipients. Examples of excipients include but are not limited to binders, diluents, adjuvants, or vehicles, such as preserving agents, fillers, polymers, disintegrating agents, glidants, welling agents, emulsifying agents, suspending agents, sweetening agents, flavoring agents, perfuming agents, lubricating agents, acidifying agents, coloring agent, dyes, preservatives and dispensing agents, or compounds of a similar nature depending on the nature of the mode of administration and dosage forms. Such ingredients, including pharmaceutically acceptable carriers and excipients that may be used to formulate oral dosage forms, are described in the Handbook of Pharmaceutical Excipients, American Pharmaceutical Association (1986), incorporated herein by reference in its entirety.

Pharmaceutically acceptable excipients are generally non-toxic to recipients at the dosages and concentrations employed and are compatible with other ingredients of the formulation. Examples of pharmaceutically acceptable excipients include water, saline, Ringer's solution, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG. Preferably the carrier is a parenteral carrier, more preferably a solution that is isotonic with the blood of the recipient.

Examples of binders include, but are not limited to, microcrystalline cellulose and cellulose derivatives, gum tragacanth, glucose solution, acacia mucilage, gelatin solution, molasses, polvinylpyrrolidine, povidone, crospovidones, sucrose and starch paste.

Examples of diluents include, but are not limited to, lactose, sucrose, starch, kaolin, salt, mannitol and dicalcium phosphate.

Examples of excipients include, but are not limited to, starch, surfactants, lipophilic vehicles, hydrophobic vehicles, pregelatinized starch, Avicel, lactose, milk sugar, sodium citrate, calcium carbonate, dicalcium phosphate, and lake blend purple. Typical excipients for dosage forms such as a softgel include gelatin for the capsule and oils such as soy oil, rice bran oil, canola oil, olive oil, corn oil, and other similar oils; glycerol, polyethylene glycol liquids, vitamin E TPGS as a surfactant and absorption enhancer (Softgels: Manufacturing Considerations; Wilkinson P, Foo Sog Hom, Special Drug Delivery Systems; Drugs and the Pharmaceutical Sciences Vol 41 Praveen Tyle Editor, Marcel Dekker 1990, 409-449; Pharmaceutical Dosage Forms and Drug Delivery by Ansel, Popovich and Allen 1995, Williams and Wilkins, Chapter 5 pp 155-225).

Examples of disintegrating agents include, but are not limited to, complex silicates, croscarmellose sodium, sodium starch glycolate, alginic acid, corn starch, potato starch, bentonite, methylcellulose, agar and carboxymethylcellulose.

Examples of glidants include, but are not limited to, colloidal silicon dioxide, talc, corn starch.

Examples of wetting agents include, but are not limited to, propylene glycol monostearate, sorbitan monooleate, diethylene glycol monolaurate and polyoxyethylene laural ether.

Examples of sweetening agents include, but are not limited to, sucrose, lactose, mannitol and artificial sweetening agents such as saccharin, and any number of spray dried flavors.

Examples of flavoring agents include, but are not limited to, natural flavors extracted from plants such as fruits and synthetic blends of compounds which produce a pleasant sensation, such as, but not limited to peppermint and methyl salicylate.

Examples of lubricants include magnesium or calcium stearate, sodium lauryl sulphate, talc, starch, lycopodium and stearic acid as well as high molecular weight polyethylene glycols.

Examples of coloring agents include, but are not limited to, any of the approved certified water soluble FD and C dyes, mixtures thereof; and water insoluble FD and C dyes suspended on alumina hydrate.

The formulation of the invention may be administered orally (e.g., in liquid form within a solvent such as an aqueous or non-aqueous liquid, or within a solid carrier), rectally, parenterally, intracisternally, intravaginally, intraperitoneally, topically (as by powders, ointments, lotion, gels, drops, transdermal patch or transcutaneous patch), bucally, in bronchial form or as an oral or nasal spray. The term “parenteral” as used herein refers to modes of administration which include intravenous (e.g., within a dextrose or saline solution), intramuscular, intrasternal, subcutaneous, intracutaneous, intrasynovial, intrathecal, periostal, intracerebroventricularly, intra-articular injection and/or infusion. Administration can be performed daily, weekly, monthly, every other month, quarterly or any other schedule of administration as a single dose injection or infusion, multiple doses, or in continuous dose form. The administration of the formulation of the present invention can be intermittent or at a gradual, continuous, constant or controlled rate to a subject. In addition, the time of day and the number of times per day that dosage form(s) is administered can vary.

The isolated two or more oligosaccharides or their variants, isomers, analogs and derivatives thereof of the invention may be also formulated into suitable pharmaceutical preparations such as solutions, suspensions, tablets, dispersible tablets, pills, capsules, powders, sustained release formulations or elixirs, for oral administration.

Oral formulations may be solid, gel or liquid. The solid dosage forms include tablets, capsules, granules, and bulk powders. Liquid formulations can, for example, be prepared by dissolving, dispersing, or otherwise mixing the isolated two or more oligosaccharides of the invention as defined above and pharmaceutical adjuvants in a carrier, such as, for example, water, saline, aqueous dextrose, glycerol, glycols, ethanol, and the like, to thereby form a solution or suspension. If desired, the formulation of the invention to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, and mannitol and dicalcium phosphate.

Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors.

Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammuoniated shellac and cellulose acetate phthalates. Film coatings include hydroxyethylcellulose, sodium carboxymethylcellulose, polyethylene glycol 4000 and cellulose acetate phthalate.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The isolated two or more oligosaccharides may be used as a prebiotic, promoting the growth of beneficial bacteria such as Lactobacillus and Bacteroides. As such, the isolated two or more oligosaccharides and their variants, isomers, analogs and derivatives may serve as a prebiotic, which selectively stimulates the growth or colonization of one or more bacterial species in the gastrointestinal tract of a host and presence of these bacteria are beneficial to the health of the host.

The two or more oligosaccharides of the invention can be derived using any number of sources and methods known to those of skill in the art. Alternatively, the two or more oligosaccharides of the invention can be synthesized by enzymatic methods, using isolated oligosaccharide biosynthetic enzyme or catabolic enzyme that participate in the biosynthesis or catabolism of two or more oligosaccharides of the invention in either forward or reverse reaction, respectively; or alternatively, two or more oligosaccharides derivatives, analogs, and variants can be derived by replacing key enzymatic steps with a different biosynthetic or catabolic enzyme and desired sugar analog to obtain the desired oligosaccharide. The two or more oligosaccharides of the invention can also be synthesized by chemical methods and purified to obtain the desired compounds.

In one embodiment, the two or more oligosaccharides and/or their derivatives, isomers, analogs and/or variants may be administered to a subject or patient.

The present invention is also directed to a kit including a container comprising the composition.

The kit comprises one or more containers with a label and/or instructions. The label can provide directions for carrying out the preparation of the two or more oligosaccharides and/or its derivatives, isomers, analogs and/or variants for example, dissolving of the dry powders, and/or treatment.

The label and/or the instructions can indicate directions for in vivo use of the formulation of the invention. The label and/or the instructions can indicate that the formulation of the invention is used alone, or in combination with another agent to treat a condition.

The label can indicate appropriate dosages for the two or more oligosaccharides and/or its derivatives, isomers, analogs and/or variants of the invention as described supra.

Suitable containers include, for example, bottles, vials, and test tubes. The containers can be formed from a variety of materials such as glass or plastic. The container can have a sterile access port (for example the container can be an intravenous solution bag or a vial having a stopper pierceable by a needle such as a hypodermic injection needle).

The present disclosure is directed to a method of increasing brain development in a patient in need thereof, comprising: providing a patient with a composition comprising two or more oligosaccharides selected from the group consisting of 2′-fucosyllactose (2′FL), 3-fucosyllactose (3FL), and 3′-sialyllactose (3′SL). The patient in need thereof can be from 5 seconds old to 18 years old, or, the patient in need thereof can be from 18 years old to 100 years old. In an embodiment, a tissue in the patient receives from about 1 to about 17,000 nmol/mL of 2′FL. In an embodiment, a tissue in the patient receives from about 1 to about 8000 nmol/mL of 3FL. In an embodiment, a tissue in the patient receives from about 1 to about 2500 nmol/mL of 3′SL.

### Examples

Twenty mother-infant pairs were recruited from maternity clinics in Los Angeles County as part of a larger cohort study, and inclusion criteria has been previously described. Briefly, mothers were included if they were ≥18 years old at delivery, had given birth to a full-term singleton newborn, were breastfeeding, and were enrolled within 1 month from delivery. Mothers were excluded if they reported medications or medical conditions that could affect physical or mental health, nutrition, or metabolism, used tobacco or recreational drugs, or had a clinical diagnosis of fetal abnormalities. The Institutional Review Board of Children's Hospital Los Angeles approved all procedures. Participants provided written informed consent prior to data collection.

Mother-infant pairs were assessed at 1-month postpartum. Historical health-related information was collected. Mothers pumped a full expression of breast milk that was analyzed for individual HMO concentrations. Infants underwent magnetic resonance imaging (MRI) scanning at approximately 1-month postpartum to assess brain tissue microstructure and regional cerebral blood flow (rCBF).

Breast milk was collected and analyzed following standard procedures. Mothers were asked to avoid eating and drinking for 1-hour and feeding or pumping breast milk for 1.5-hours before collection. Mothers were encouraged to pump the entire contents of a single breast expression to ensure collection of fore, mid, and hind milk to standardize milk collection as much as possible. Aliquots were stored at −80° C. until HMO characterization at the University of California San Diego. Raffinose was added to each sample as an internal standard for absolute quantification. HMOs were isolated with high-throughput solid-phase extraction, fluorescently labeled, and measured using HPLC. Nineteen HMOs were quantified based on standard retention times and mass spectrometric analysis, which accounted for >90% of total HMO composition. The initial focus, however, was on the most abundant fucosylated and sialylated HMOs reported to influence brain development in animals and infants: 2′FL, 3FL, 3′SL, and 6′SL. Secretor status was defined by the presence or near absence of HMOs 2′FL or lacto-N-fucopentaose I.

MRI data were collected on a 3 Tesla Philips Achieva MRI scanner equipped with a 32-channel receive-only head coil. All data were acquired without sedation or contrast agent. Mothers and staff lulled infants to sleep with feeding and swaddling. Scans were visually monitored and repeated for any visible motion. Detailed image quality was assessed during preprocessing within 48 hours of scan acquisition.

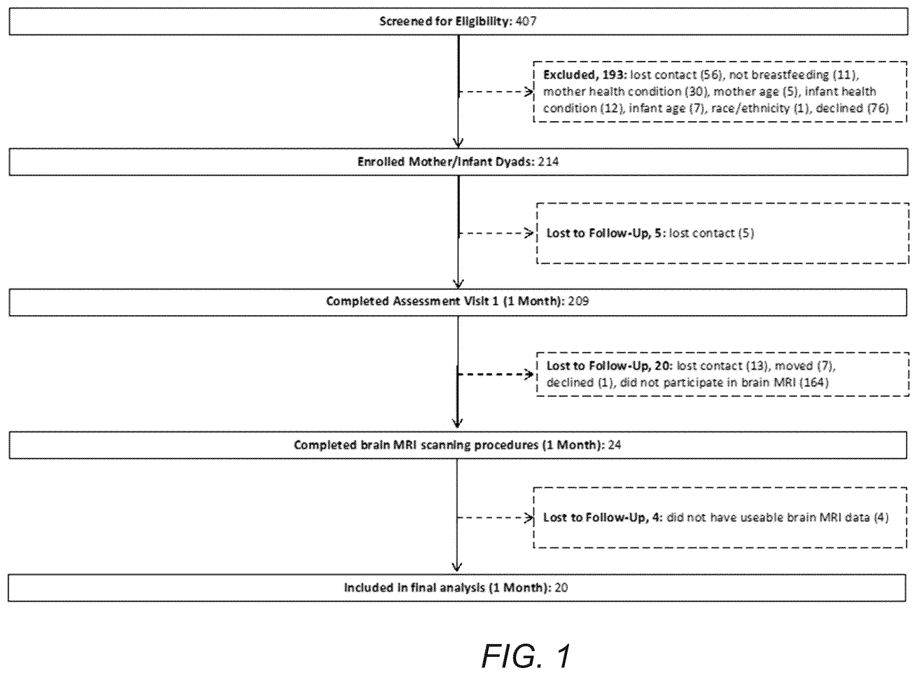

Diffusion Tensor Imaging (DTI), which measures the diffusion of water as influenced by characteristics of brain tissue microstructure: The template brain was selected using a 2-step procedure to find the brain that was morphologically representative of all brains in our sample, as described in FIG. 1. Quality assurance procedures were instituted to exclude datasets with excessive motion. The diffusion tensor (D) was estimated at each voxel of the pre-processed DTI data in DSI Studio (RRID:SCR_009557) and computed the scalar indices fractional anisotropy (FA), mean diffusivity (MD), axial diffusivity (AD), and radial diffusivity (RD). These maps were edited to remove nonbrain tissue and then were threshed to remove background noise. Edited MD map was used to mask out nonbrain tissue in the FA, AD, and RD maps. The edited maps for each infant were coregistered using a rigid body transformation (3 translations and 3 rotations) to the infant's T1-weighted (T1w) anatomical image, which were in turn used as an intermediary source to coregister the maps for each infant to the final template brain. The FA, AD, MD, and RD maps were then smoothed using a Gaussian kernel with FWHM=4 mm.

Arterial Spin Labeling (ASL), which quantifies rCBF, a surrogate measure of cellular metabolism: The rCBF brain images and the Mo_wM image to the first rCBF image for each participant were aligned in native imaging space to correct for head motion. Images with >0.5 mm inter-frame motion were excluded from further processing. The coregistered rCBF images were spatially smoothed using a Gaussian kernel of FWHM=4 mm to improve the signal-to-noise ratio while avoiding loss of spatial precision in locating our effects of interest. A brain mask was generated for each infant using the mean rCBF image. An in-house software was used to construct for each infant a voxel-wise map of rCBF from the ASL time series and Mo_wm image: 1) pair-wise subtracted the control images from the labeled images; 2) Perfusion-weighted images were calculated by pair-wise subtraction of the control and labeled images, followed by averaging across the imaging time series. The perfusion map (rCBF) was calculated using a single compartment model. A linear transformation with 6 degrees of freedom was used to coregister the quantitative rCBF maps for each infant to their T1-weighted anatomical images, which were in turn used as an intermediary source to coregister the rCBF images for each infant to the final brain template.

Descriptive statistics are presented as mean±standard deviation (SD) for continuous variables and as frequency (percentage) for categorical variables. Normal distribution and homogeneity of variances were confirmed by Shapiro-Wilks W and Levene's tests. Values >3 SD from the mean were identified as outliers. Multiple linear regression was applied to each voxel of the image to assess the significance of the correlation coefficient for the concentration (nmol/mL) of each HMO (2′FL, 3FL, 3′SL, 6′SL) and total HMO-bound Sia with FA, MD, and rCBF values. The significance was assessed in the first in the total cohort (N=20) and then in infants of secretor mothers only (N=17), as secretor mothers have an active secretor locus that encodes for a functional fucosyltransferase 2 enzyme and produces higher concentrations of alpha-1-2-fucosylated HMOs like 2′FL relative to those classified as non-secretors. Pre-pregnancy BMI, postmenstrual age (PMA) at time of MRI scan, birthweight, and sex were included as covariates in all analyses. We used the Benjamini-Yekutieli procedure for False Discovery Rate to correct P-values for the number of statistical comparisons in the regressions modeling each HMO exposure variable. Corrected P-values were color-coded and displayed on the template brain. All statistical maps were constructed using in-house software. Regions of interest were identified using an age-specific DTI atlas for the infant brain. With a given sample size of 20, alpha-level of 0.05, and medium effect size for correlation coefficient, the study had a power of 0.69 to detect real effects (G*Power, version 3.1.9.6).

Results: Characteristics of mother-infant dyads are shown in Table 1. Of the participants enrolled in the larger study, 20 mothers whose infants had usable MRI data at 1-month of age were included in the analysis (FIG. 1). All mothers self-identified as Hispanic. By design, infants were born full-term with normal birthweight (i.e., ≥2,500 grams). Proportions of males and females were similar. Mothers reported 7.1±1.6 feedings per day, defined as exclusive milk feeding for infants at 1-month of age. Eighty-five percent of mothers were HMO secretors.

TABLE 1
Characteristics of participants and their infants.

| Variable | Mean | SD | Min. | Max. |
|---|---|---|---|---|
| Mothers | | | | |
| Age at delivery (years) | 28.5 | 6.74 | 18 | 40 |
| Pre-pregnancy BMI (kg/m²) | 27.8 | 6.25 | 18.7 | 42.5 |
| Secretor status (%) | 85.0 | | | |
| Education level (%) | | | | |
| Less than high school | 20.8 | | | |
| Completed high school | 58.4 | | | |
| Completed college | 20.8 | | | |
| Infants | | | | |
| Female (%) | 62.5 | | | |
| Birthweight (g) | 3,360 | 498 | 2,300 | 4,510 |
| Gestational age at birth (days) | 47.9 | 7.06 | 38.0 | 65.0 |
| Postmenstrual age at MRI | 323 | 10.5 | 300 | 351 |
| scanning (days) | | | | |
| Breast feedings per day, | 7.08 | 1.64 | 1 | 8 |
| 1-month (number) | | | | |

Values are mean ± SD or %. Postmenstrual age at the time of MRI scanning is defined as the time that elapsed between the mother's last menstrual period and birth of the infant (i.e., gestational age at birth) plus chronological age of the infant from birth to the time of the MRI scan.

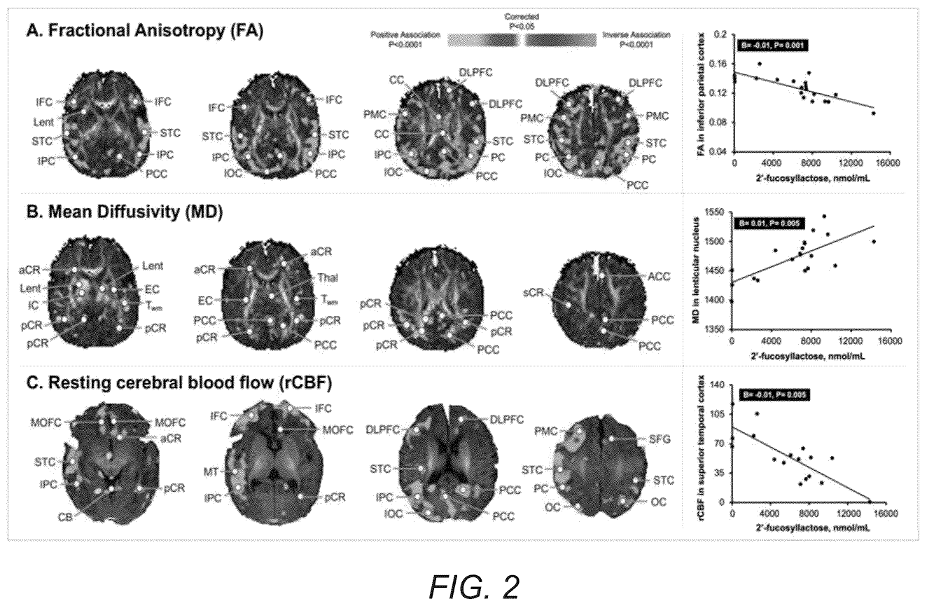

HMO 2′FL exposure at 1-month of age and newborn MRI measures: Overall, exposure to individual HMOs associated significantly with MRI measures of tissue microstructure and rCBF in the infant brain at 1-month of age. Because associations of fucosylated and sialylated HMOs with neuroimaging indices for infants in the total sample were the same as associations of these HMOs with neuroimaging indices for infants of secretor mothers only, results for infants in the total sample were presented. Exposure to 2′FL was associated with reduced FA values throughout the cortex and increased MD values in posterior cortical gray matter (e.g., right posterior cingulate cortex, PCC; B=0.02, p=0.001), posterior white matter (e.g., left posterior corona radiata, pCR; B=0.02, p=0.003), and subcortical gray matter nuclei (e.g., left lenticular nucleus; B=0.01, p=0.005) (FIG. 2). Exposure to 2′FL was associated with reduced rCBF in cortical gray matter of the frontal, temporal, parietal, and occipital lobes (e.g., superior temporal cortex; B=−0.01, p=0.005) (FIG. 2).

FIG. 2 shows the statistical significance of the associations of 2′FL exposure with measures of brain tissue microstructure and rCBF at each point on the surface of the brain was color-coded, with warm colors representing significant positive associations and cool colors representing significant inverse associations. Only P-values that survived FDR correction are plotted. A) Concentrations of 2′FL associated inversely with FA values throughout much of the cortical mantle. The scatterplot highlights the significant inverse association of 2′FL exposure with FA values located in the right inferior parietal cortex (IPC). B) Concentrations of 2′FL associated positively with MD values in posterior gray matter, posterior white matter, and subcortical gray matter nuclei. The scatterplot highlights the significant positive association of 2′FL exposure with MD values located in the Lent. C) Concentrations of 2′FL associated inversely with rCBF values in cortical gray matter in similar locations as FA values. The scatterplot highlights the significant inverse association of 2′FL exposure with rCBF values located in the left superior temporal cortex (STC).

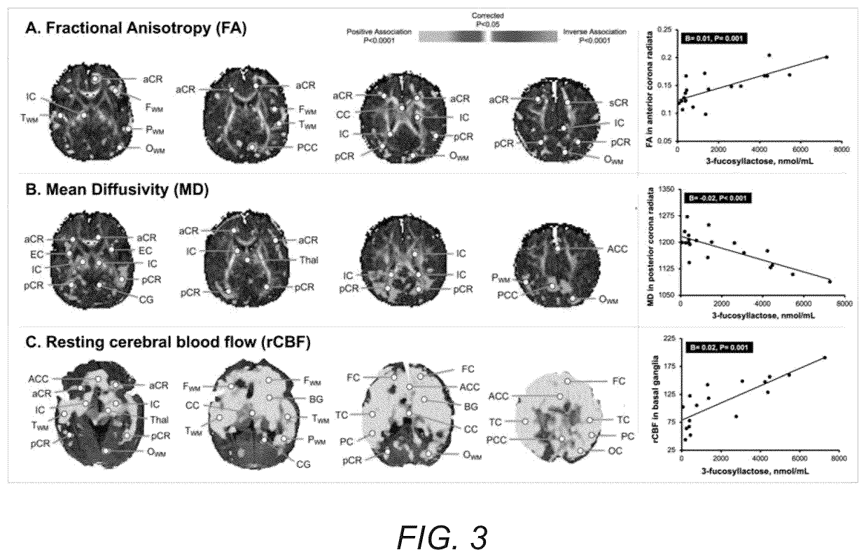

HMO 3FL exposure at 1-month of age and newborn MRI measures: Exposure to 3FL was associated with increased FA values in white matter throughout the frontal, temporal, parietal, and occipital lobes, but particularly in the left internal capsule (IC) and right anterior corona radiata (aCR) (B=0.01, p=0.001) (FIG. 3). Exposure to 3FL was associated with reduced MD values in similar regions (e.g., left IC; B=−0.01, p=0.007) and especially in posterior white matter (e.g., left pCR; B=−0.02, p<0.001). In addition, exposure to 3FL was associated with increased rCBF in the cortex of most of the brain and within white matter of the frontal, temporal, parietal, and occipital lobes (e.g., right occipital white matter; B=0.01, p=0.001) and subcortical gray matter nuclei (e.g., right basal ganglia; B=0.02, p=0.001).

FIG. 3 shows the statistical significance of the associations of 3FL exposure with measures of brain tissue microstructure and rCBF at each point on the surface of the brain is color-coded, with warm colors representing significant positive associations and cool colors representing significant inverse associations. Only P-values that survived FDR correction are plotted. A) Concentrations of 3FL associated positively with FA values in developing white matter. The scatterplot highlights the significant positive association of 3FL exposure with FA values in the right aCR. B) Concentrations of 3FL associated inversely with MD values in similar regions of developing white matter as FA values. The scatterplot highlights the significant inverse association of 3FL exposure with MD values in the right pCR. C) Concentrations of 3FL associated positively with rCBF values throughout much of the cortex and in developing white matter. The scatterplot highlights the significant positive association of 3FL exposure with rCBF values in the basal ganglia (BG).

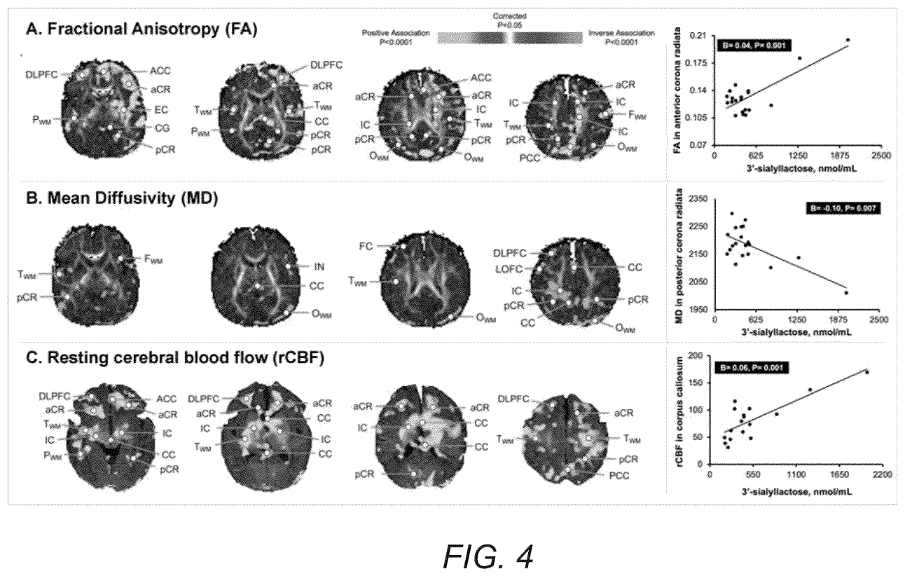

HMO 3′SL exposure at 1-month of age and newborn MRI measures: Exposure to the sialylated HMO, 3′SL, was associated with increased FA values in white matter throughout the brain (FIG. 4), particularly in the splenium of the corpus callosum (CC), posterior corona radiata (pCR), and anterior corona radiata (aCR) (e.g., right aCR; B=0.04, p=0.001), and in cortical gray matter of the frontal lobe, including the anterior cingulate cortex (ACC) and dorsolateral prefrontal cortex (DLPFC) (e.g., right DLPFC; B=0.03, p=0.001). Exposure to 3′SL was associated with reduced MD values in similar regions, particularly in posterior white matter (e.g., left pCR; B=−0.10, p=0.007). Exposure to 3′SL was associated with increased rCBF in white matter bilaterally, including the IC (B=0.04, p<0.001), CC (B=0.06, p=0.001), and aCR (B=0.07, p<0.001), and in cortical gray matter of the frontal lobe (DLPFC, ACC). In the total sample, the 3′SL value for one participant was identified as a statistical outlier. However, the participant was included in the analysis because the 3′SL value was biologically plausible based on previous reports.

FIG. 4 shows the statistical significance of the associations of 3′SL exposure with measures of brain tissue microstructure and rCBF at each point on the surface of the brain is color-coded, with warm colors representing significant positive associations and cool colors representing significant inverse associations. Only P-values that survived FDR correction are plotted. A) Concentrations of 3′SL associated positively with FA values in developing white matter throughout much of the brain and in cortical gray matter of the frontal lobe. The scatterplot highlights the significant positive association of 3′SL exposure with FA values in the right aCR. B) Concentrations of 3′SL associated inversely with MD values in posterior white matter regions. The scatterplot highlights the significant inverse association of 3′SL exposure with MD values in the left pCR. C) Concentrations of 3′SL associated positively with rCBF values in white matter pathways bilaterally and cortical gray matter of the frontal lobe, and findings were in similar locations as associations of 3′SL with FA values. The scatterplot highlights the significant positive association of 3′SL exposure with rCBF values in the CC.

Sia exposure at 1-month of age and newborn MRI measures: Similar findings were observed for associations of total HMO-bound Sia exposure with MRI measures. Exposure to total HMO-bound Sia was associated with increased FA values in white matter throughout the brain and in cortical gray matter of the DLPFC, ACC, and PCC, and with reduced MD values in posterior white matter and subcortical gray matter nuclei. Exposure to total HMO-bound Sia was associated with increased rCBF in similar white matter regions, including the IC and CC, and in gray matter in the frontal lobe (DLPFC, ACC). In contrast, exposure to the structural isomer 6′SL was not significantly associated with any MRI measures. The data show that individual HMO exposures differentially associate with MRI indices of newborn brain tissue organization and rCBF, which reflect structural and metabolic characteristics that foster future cognitive and behavioral functions. There are specific roles of 2′FL, 3FL, and 3′SL in promoting maturation of cortical gray matter and developing white matter, which in turn may guide recommendations for the nutritional care of infants and supplementation strategies that supports optimal brain development. These findings by the inventors are also published in an article entitled “Associations of Human Milk Oligosaccharides with Infant Brain Tissue Organization and Regional Blood Flow at 1 Month of Age”, Berger et al., Nutrients, 2022, 14, 3820, https://doi.org/10.3390/nu14183820, the disclosure of which is hereby incorporated by reference in its entirety for all purposes.

From the foregoing description, those skilled in the art can appreciate that the present teachings can be implemented in a variety of forms. Therefore, while these teachings have been described in connection with particular embodiments and examples thereof, the true scope of the present teachings should not be so limited. Various changes and modifications can be made without departing from the scope of the teachings herein.

This scope disclosure is to be broadly construed. It is intended that this disclosure disclose equivalents, means, systems and methods to achieve the devices, activities and mechanical actions disclosed herein. For each device, article, method, mean, mechanical element or mechanism disclosed, it is intended that this disclosure also encompass in its disclosure and teaches equivalents, means, systems and methods for practicing the many aspects, mechanisms and devices disclosed herein. The claims of this application are likewise to be broadly construed. The description of the inventions herein in their many embodiments is merely exemplary in nature and, thus, variations that do not depart from the gist of the invention are intended to be within the scope of the invention. Such variations are not to be regarded as a departure from the spirit and scope of the invention.

What is claimed is:

1. A composition, comprising:
two or more oligosaccharides selected from the group consisting of 2′-fucosyllactose (2′FL), 3-fucosyllactose (3FL), and 3′-sialyllactose (3′SL);
wherein the composition is a non-naturally occurring formulation.

2. The composition of claim 1, further comprising 6′-sialyllactose (6′SL).

3. The composition of claim 1, further comprising at least one additional nutrient selected from the group consisting of other carbohydrates, protein, fat, mineral, vitamin, and combinations thereof.

4. The composition of claim 1, wherein the composition is in a form of a powder.

5. The composition of claim 1, wherein the composition is in a form of a liquid.

6. A kit comprising a container including the composition of claim 1.

7. A method of increasing brain development in a patient in need thereof, comprising:
providing a patient with a composition comprising two or more oligosaccharides selected from the group consisting of 2′-fucosyllactose (2′FL), 3-fucosyllactose (3FL), and 3′-sialyllactose (3′SL);
wherein the composition is a non-naturally occurring formulation.

8. The method of claim 7, wherein the patient in need thereof is from 5 seconds old to 18 years old.

9. The method of claim 7, wherein the patient in need thereof is from 18 years old to 100 years old.

10. The method of claim 7, wherein a tissue in the patient receives from about 1 to about 17,000 nmol/mL of 2′FL.

11. The method of claim 7, wherein a tissue in the patient receives from about 1 to about 8000 nmol/mL of 3FL.

12. The method of claim 7, wherein a tissue in the patient receives from about 1 to about 2500 nmol/mL of 3′SL.
